(19) United States
(12) Patent Application Publication
(10) Pub. No.: US 2018/0353082 A1
(43) Pub. Date: Dec. 13, 2018

(54) PHOTOACOUSTIC APPARATUS AND OBJECT INFORMATION ACQUIRING METHOD

(71) Applicant: CANON KABUSHIKI KAISHA, Tokyo (JP)

(72) Inventor: Naoto Abe, Machida-shi (JP)

(21) Appl. No.: 15/992,539

(22) Filed: May 30, 2018

(30) Foreign Application Priority Data
Jun. 9, 2017 (JP) 2017-114434

(51) Int. Cl. A61B 5/00; G01N 29/24; G01N 29/28; G01N 29/44; G01N 29/06; A61B 5/145

(52) U.S. Cl. CPC A61B 5/0095; G01N 29/2418; G01N 29/28; G01N 29/4472; A61B 2576/00; A61B 5/14542; A61B 5/7203; G01N 2291/02466; G01N 29/0672

(57) ABSTRACT

An object of the present invention is to increase freedom of selection of light sources in a photoacoustic apparatus. A photoacoustic apparatus includes: a light source which irradiates an object with beams of light of a plurality of wavelengths either simultaneously or at different time points; acoustic wave detecting means for receiving an acoustic wave generated at the object due to the irradiated beams of light, and converting the acoustic wave into an electrical signal; and signal processing means for acquiring characteristics information of the object based on at least the electrical signal.

### BACKGROUND OF THE INVENTION

### Field of the Invention

The present invention relates to an apparatus for acquiring information of an object using a photoacoustic effect.

### Description of the Related Art

Recently, in the field of medicine, research is underway on imaging of structural information and physiological information or, in other words, functional information of the inside of an object. Photoacoustic tomography (PAT) is recently being proposed as such an imaging technique.

When a living organism that is an object is irradiated with light such as laser light, an acoustic wave (typically, an ultrasonic wave) is generated when the light is absorbed by living tissue inside the object. This phenomenon is referred to as a photoacoustic effect and an acoustic wave generated by the photoacoustic effect is referred to as a photoacoustic wave. Since tissues constituting an object have respectively different absorption rates of optical energy, sound pressure of generated photoacoustic waves also differs. With PAT, by receiving a generated photoacoustic wave with a probe and mathematically analyzing a received signal, characteristic information of the inside of an object can be acquired.

Photoacoustic apparatuses capable of readily accessing an observation site using a hand-held probe in a similar manner to ultrasonic diagnostic apparatuses are being researched and developed.

Japanese Patent Application Laid-open No. 2015-142740 discloses a photoacoustic apparatus which individually irradiates light of a first wavelength in which absorption coefficients of oxyhemoglobin and deoxyhemoglobin are equal to each other and light of a second wavelength that differs from the first wavelength to obtain a blood vessel position (structural information) and a biological characteristic distribution (functional information).

In addition, Japanese Patent Application Laid-open No. 2017-006161 discloses a method of improving an S/N ratio by respectively irradiating an object a plurality of times with light of a first wavelength and light of a second wavelength and adding up obtained photoacoustic signals for each wavelength.

When acquiring functional information such as oxygen saturation using a photoacoustic apparatus, an object must be irradiated with beams of light of a plurality of wavelengths that are respectively readily absorbed by oxyhemoglobin and deoxyhemoglobin. In addition, when acquiring structural information such as a blood vessel structure, the wavelength of light to be irradiated is favorably a wavelength with equal absorption coefficients with respect to oxyhemoglobin and deoxyhemoglobin.

As described above, a photoacoustic apparatus requires a light source capable of generating light of a prescribed wavelength in order to obtain desired information. However, limiting wavelengths means that options regarding light sources are narrowed down. In particular, in cases of small apparatuses such as a hand-held probe, a semiconductor light-emitting element such as a semiconductor laser or a light-emitting diode which is a small light source may be used.

However, a semiconductor light-emitting element does not necessarily produce required light output when emitting the prescribed wavelength. In other words, there is a problem in that, due to constraints on the wavelength of light to be emitted, a light source cannot always be selected while prioritizing light output.

The present invention has been made in consideration of such problems existing in prior art and an object thereof is to increase freedom of selection of light sources in a photoacoustic apparatus.

### SUMMARY OF THE INVENTION

The present invention provides a photoacoustic apparatus, comprising:

a light source configured to simultaneously irradiate an object with lights of a plurality of wavelengths;

acoustic wave detecting unit configured to receive acoustic waves generated from the object being simultaneously irradiated with the lights of the plurality of wavelengths, and convert the acoustic waves into electrical signals; and

signal processing unit configured to acquire characteristics information of the object based on at least the electrical signals.

The present invention provides a photoacoustic apparatus, comprising:

a light source configured to respectively irradiate an object with lights of a plurality of wavelengths at different time points;

acoustic wave detecting unit configured to convert a plurality of acoustic waves generated from the object being irradiated with the lights of the plurality of wavelengths, into a plurality of electrical signals; and

signal processing unit configured to acquire characteristics information of the object based on at least a combined electrical signal acquired by combining the plurality of electrical signals.

Further features of the present invention will become apparent from the following description of exemplary embodiments with reference to the attached drawings.

### BRIEF DESCRIPTION OF THE DRAWINGS

FIG. 1A is a graph showing absorption coefficients of oxyhemoglobin and deoxyhemoglobin;

FIG. 1B is a graph showing absorption coefficients of oxyhemoglobin and deoxyhemoglobin;

FIG. 1C is a graph showing absorption coefficients of oxyhemoglobin and deoxyhemoglobin;

FIG. 2 is a block diagram of a photoacoustic apparatus according to a first embodiment;

FIG. 3 is a diagram for explaining a structure of a probe according to the first embodiment;

FIG. 4 is a configuration diagram of a computer according to the first embodiment;

FIG. 5 is a timing chart according to the first embodiment;

FIG. 6 is a timing chart according to a second embodiment;

FIG. 7 is a graph showing absorption coefficients of oxyhemoglobin and deoxyhemoglobin; and

FIG. 8 is a timing chart according to a third embodiment.

### DESCRIPTION OF THE EMBODIMENTS

Hereinafter, embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the embodiments described below.

The present invention relates to a technique for detecting an acoustic wave propagating from an object and generating and acquiring characteristic information of the inside of the object. Accordingly, the present invention can be applied to a photoacoustic apparatus (an object information acquiring apparatus) or a control method thereof, or an object information acquiring method. The present invention can also be applied to a program that causes an information processing apparatus including hardware resources such as a CPU and a memory to execute these methods or a computer-readable non-transitory storage medium storing the program.

The photoacoustic apparatus (the object information acquiring apparatus) according to the present invention is an apparatus utilizing a photoacoustic effect in which an acoustic wave generated inside an object by irradiating the object with light (an electromagnetic wave) is received and characteristic information of the object is acquired as image data. In this case, characteristic information refers to information on a characteristic value corresponding to each of a plurality of positions inside the object which is generated using a received signal obtained by receiving a photoacoustic wave.

Characteristic information acquired by photoacoustic measurement is a value reflecting an absorption rate of optical energy. For example, characteristic information includes a generation source of acoustic waves generated by light irradiation, initial sound pressure inside an object, an optical energy absorption density or an absorption coefficient derived from initial sound pressure, and a concentration of substances constituting tissue.

In addition, a distribution of oxygen saturation can be calculated by obtaining an oxyhemoglobin concentration and a deoxyhemoglobin concentration as concentrations of substances. Furthermore, a glucose concentration, a collagen concentration, a melanin concentration, a volume fraction of fat or water, and the like can also be obtained. Moreover, substances with a characteristic light absorption spectrum including contrast agents administered into the body can be the targets of characteristic information acquisition.

A two-dimensional or three-dimensional characteristic information distribution is obtained based on characteristic information at each position in the object. Distribution data may be generated as image data. Characteristic information may be obtained as distribution information of respective positions inside the object instead of as numerical data. In other words, distribution information such as an initial sound pressure distribution, an energy absorption density distribution, an absorption coefficient distribution, and an oxygen saturation distribution may be obtained.

An acoustic wave in the present specification is typically an ultrasonic wave and includes an elastic wave which is also referred to as a sonic wave or a photoacoustic wave. An electrical signal transformed from an acoustic wave by a probe or the like is also referred to as an acoustic signal. However, descriptions of an ultrasonic wave and an acoustic wave in the present specification are not intended to limit a wavelength of the elastic waves. An acoustic wave generated by a photoacoustic effect is referred to as a photoacoustic wave or an optical ultrasonic wave. An electrical signal derived from a photoacoustic wave is also referred to as a photoacoustic signal. It should be noted that, in the present specification, a photoacoustic signal is a concept encompassing both analog signals and digital signals. Distribution data is also referred to as photoacoustic image data or reconstructed image data.

The present invention is an invention which involves, instead of acquiring a photoacoustic signal based on a photoacoustic wave generated by irradiating light of a desired wavelength, irradiating beams of light at a plurality of wavelengths that differ from the desired wavelength and acquiring a signal equivalent to a desired photoacoustic signal based on a generated photoacoustic signal.

In this case, the term equivalent need not necessarily mean being the same. Cases in which a difference is small enough to be negligible in biological diagnosis can be considered being equivalent.

Moreover, when a light source of light to be irradiated on an object is a laser, a “wavelength” according to the present embodiment can be a peak wavelength or an oscillation wavelength. Alternatively, when using a light source that emits light with a wider wavelength band than a laser such as a laser diode (LED), a “wavelength” can be a central wavelength.

### Outline of Embodiments

A photoacoustic apparatus according to an embodiment of the present invention includes: a light source which simultaneously irradiates an object with beams of light of a plurality of wavelengths; and acoustic wave detecting means for receiving an acoustic wave generated due to the object being simultaneously irradiated with the beams of light of a plurality of wavelengths, and converting the acoustic wave into an electrical signal. The photoacoustic apparatus according to an embodiment of the present invention further includes signal processing means for acquiring characteristics information of the object based on at least an output electrical signal converted from an acoustic wave.

Let us now assume that an object includes a plurality of light absorbers each having a different wavelength dependency of an absorption coefficient. When a wavelength at which respective absorption coefficients of the plurality of light absorbers become equal to each other is defined as a reference wavelength, irradiating the object with light of the reference wavelength enables distribution information and the like of the light absorbers included in the object as well as characteristics information of the object to be obtained. In the present embodiment, wavelengths that differ from the reference wavelength are selected as the plurality of wavelengths described above. Specifically, when the object includes a first light absorber and a second light absorber each having a different wavelength dependency of an absorption coefficient, a plurality of wavelengths satisfying the following conditions are selected.

That is, the plurality of wavelengths are a combination of wavelengths such that a ratio of a sum of absorption coefficients of the first light absorber to a sum of absorption coefficients of the second light absorber at each of the plurality of wavelengths (a first ratio) becomes equal to a ratio of the absorption coefficient of the first light absorber to the absorption coefficient of the second light absorber at the reference wavelength (a second ratio). By selecting a plurality of wavelengths which satisfy the above, photoacoustic data equivalent to photoacoustic data obtained by irradiating the object with the reference wavelength is obtained.

In addition, there may be a difference between the ratio of a sum of absorption coefficients of the first light absorber to a sum of absorption coefficients of the second light absorber at each of the plurality of wavelengths (the first ratio) and the ratio of the absorption coefficient of the first light absorber to the absorption coefficient of the second light absorber at the reference wavelength (the second ratio). In this case, correcting means for correcting the difference is used. For example, photoacoustic data (an electrical signal) equivalent to photoacoustic data obtained by irradiating the object with the reference wavelength is obtained by adjusting a sum of irradiation times of each of the beams of light of the plurality of wavelengths or adjusting an irradiated light amount of each of the beams of light of the plurality of wavelengths.

When the object is not simultaneously irradiated with beams of light of a plurality of wavelengths, a photoacoustic apparatus such as that described below can be used. Specifically, a photoacoustic apparatus can be used which includes: a light source which simultaneously irradiates an object with beams of light of a plurality of wavelengths at different time points; and acoustic wave detecting means for converting a plurality of acoustic waves generated due to the object being irradiated with the beams of light of a plurality of wavelengths, into a plurality of electrical signals. The photoacoustic apparatus further includes signal processing means for acquiring characteristics information of the object based on at least a combined electrical signal acquired by combining the plurality of electrical signals. In other words, even when the beams of light of a plurality of wavelengths are irradiated at mutually different time points, by combining electrical signals respectively corresponding to the beams of light of a plurality of wavelengths, photoacoustic data (an electrical signal) equivalent to a case where the beams of light of the plurality of wavelengths are simultaneously irradiated is obtained. Moreover, the combined electrical signal is favorably acquired by performing an averaging process of the plurality of electrical signals.

Hereinafter, apparatus configurations described in the outline of embodiments presented above will be described in detail. In addition, oxyhemoglobin and deoxyhemoglobin will be described as an example of light absorbers.

### First Embodiment

### Outline of Apparatus

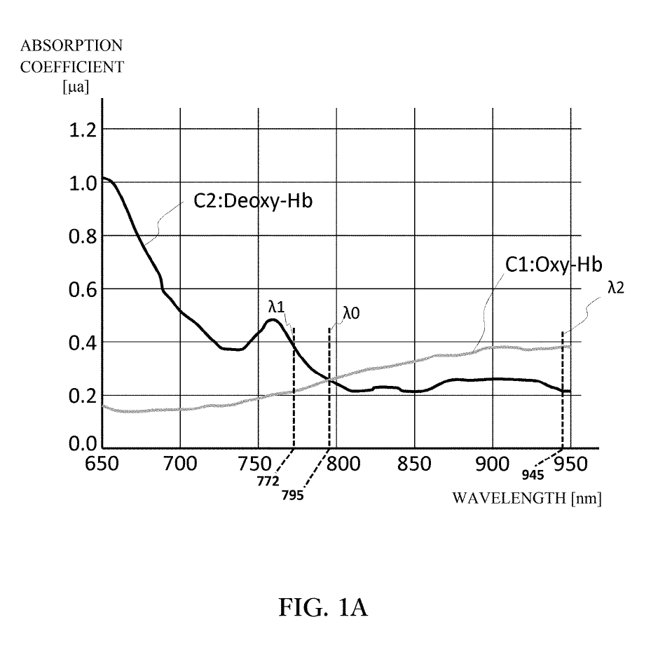

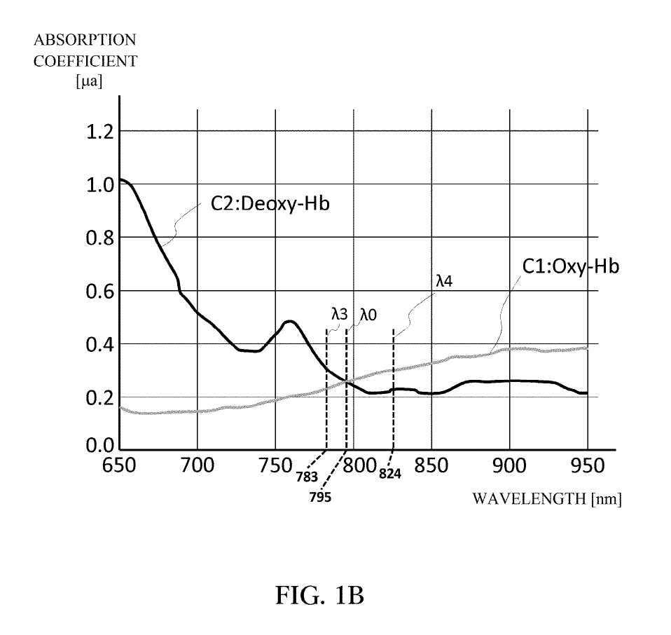

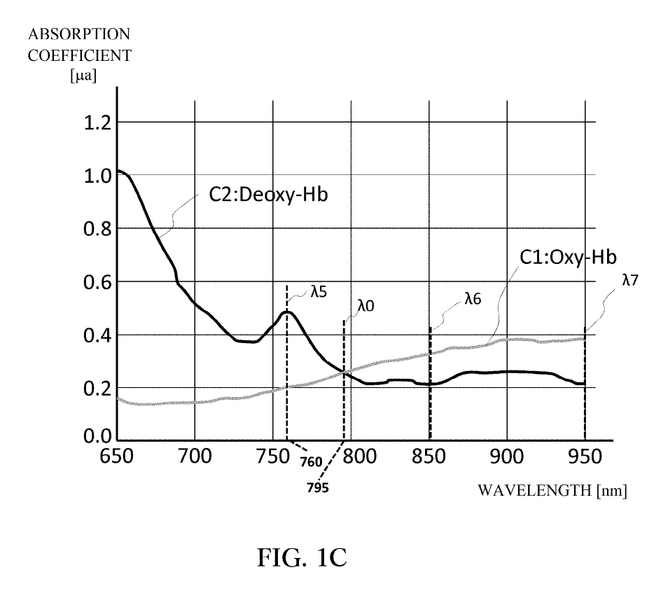

Problems solved by a photoacoustic apparatus according to an embodiment of the present invention will be described with reference to FIGS. 1A to 1C.

FIG. 1A is a diagram showing a relationship between wavelengths of light to be irradiated to an object and absorption coefficients of light absorbers present inside the object. Moreover, C1 in the diagram denotes an absorption coefficient of oxyhemoglobin (oxygenated hemoglobin) and C2 denotes an absorption coefficient of deoxyhemoglobin (reduced hemoglobin).

As is apparent from the diagram, at a wavelength λ0 (approximately 795 nm), absorption coefficients of oxyhemoglobin and deoxyhemoglobin become equal to each other. Therefore, by irradiating the object with light of the wavelength λ0 and acquiring a photoacoustic signal, structural information of blood vessels (arteries and veins) can be obtained with good accuracy.

In a first embodiment, a signal equivalent to a photoacoustic signal obtained by irradiating an object with light of the wavelength λ0 is acquired without using a light source having the wavelength λ0.

In the first embodiment, the wavelength λ0 will be referred to as a desired wavelength (a reference wavelength). In addition, as will be described later, by causing a light source to generate beams of light of a plurality of wavelengths that differ from the desired wavelength, a photoacoustic signal equivalent to a photoacoustic signal (hereinafter, a desired signal) obtained by irradiating light of the desired wavelength is obtained. Moreover, when oxyhemoglobin and deoxyhemoglobin are measurement targets, the reference wavelength can be set to a wavelength at which respective absorption coefficients become equal to each other such as from 790 nm to 800 nm.

In FIG. 1A, a wavelength λ1 and a wavelength λ2 are wavelengths which differ from the desired wavelength and at which magnitude relationships between absorption coefficients of oxyhemoglobin and deoxyhemoglobin are respectively inverted. In the present embodiment, it is assumed that two wavelengths are selected such that the absorption coefficient of oxyhemoglobin at the wavelength λ1 is equal to the absorption coefficient of deoxyhemoglobin at the wavelength λ2 and the absorption coefficient of deoxyhemoglobin at the wavelength λ1 is equal to the absorption coefficient of oxyhemoglobin at the wavelength λ2.

For example, let us assume that the wavelength λ1 is 772 nm and the wavelength λ2 is 945 nm. When beams of light of the two wavelengths are simultaneously irradiated to the object at the same output, sound pressure of a photoacoustic wave generated due to oxyhemoglobin takes a value in accordance with a sum of an absorption coefficient at the wavelength λ1 of oxyhemoglobin and an absorption coefficient at the wavelength λ2 of oxyhemoglobin. In addition, sound pressure of a photoacoustic wave generated due to deoxyhemoglobin takes a value in accordance with a sum of an absorption coefficient at the wavelength λ1 of deoxyhemoglobin and an absorption coefficient at the wavelength λ2 of deoxyhemoglobin.

In other words, by simultaneously irradiating beams of light of the wavelengths λ1 and λ2 and acquiring acoustic waves, a photoacoustic signal equivalent to a photoacoustic signal (a desired signal) generated when irradiating light of the wavelength λ0 can be obtained.

Moreover, while a wavelength (λ0) at which absorption coefficients of oxyhemoglobin and deoxyhemoglobin are the same has been described as the desired wavelength in the present example, the desired wavelength may be another wavelength. Even in this case, by simultaneously irradiating beams of light having a plurality of wavelengths that differ from the desired wavelength in a similar manner to a case where the desired wavelength is the wavelength λ0, a photoacoustic signal equivalent to the desired signal can be obtained.

In addition, while the wavelengths λ1 and λ2 have been described in the present example as the plurality of wavelengths that differ from the desired wavelength, a combination of other wavelengths can also be used. For example, wavelengths λ3 and λ4 may be selected as shown in FIG. 1B. In the example shown in FIG. 1B, the wavelength λ3 is set to 783 nm and the wavelength λ4 is set to 824 nm. In the present example, there is a similar relationship in which the absorption coefficient of oxyhemoglobin at the wavelength λ3 is equal to the absorption coefficient of deoxyhemoglobin at the wavelength λ4 and the absorption coefficient of deoxyhemoglobin at the wavelength λ3 is equal to the absorption coefficient of oxyhemoglobin at the wavelength λ4.

By selecting wavelengths that satisfy this relationship, a photoacoustic signal equivalent to a desired signal obtained by irradiating light of the desired wavelength can be obtained.

Furthermore, three wavelengths may be selected as wavelengths that differ from the desired wavelength λ0.

In FIG. 1C, a wavelength λ5 is set to 760 nm, a wavelength λ6 is set to 850 nm, and a wavelength λ7 is set to 950 nm. In the present example, a sum of an absorption coefficient at the wavelength λ5, an absorption coefficient at the wavelength λ6, and an absorption coefficient at the wavelength λ7 of oxyhemoglobin is equal to a sum of an absorption coefficient at the wavelength λ5, an absorption coefficient at the wavelength λ6, and an absorption coefficient at the wavelength λ7 of deoxyhemoglobin.

When beams of light of the three wavelengths are simultaneously irradiated at the same output, sound pressure of a photoacoustic wave generated due to oxyhemoglobin takes a value in accordance with a sum of the absorption coefficient at the wavelength λ5, the absorption coefficient at the wavelength λ6, and the absorption coefficient at the wavelength λ7 of oxyhemoglobin. In a similar manner, sound pressure of a photoacoustic wave generated due to deoxyhemoglobin takes a value in accordance with a sum of the absorption coefficient at the wavelength λ5, the absorption coefficient at the wavelength λ6, and the absorption coefficient at the wavelength λ7 of deoxyhemoglobin.

In this manner, in the first embodiment, a photoacoustic signal equivalent to a desired signal can be obtained by simultaneously irradiating beams of light of wavelengths that differ from the desired wavelength from a plurality of light sources.

Moreover, timings at which beams of light are emitted from the plurality of light sources favorably satisfy the following conditions.

When beams of light are not simultaneously irradiated from a plurality of light sources, corresponding photoacoustic waves are also generated at mutually different time points. In addition, photoacoustic waves respectively generated at different time points are combined and converted into a photoacoustic signal. In other words, the obtained photoacoustic signal has a rounded waveform as though passed through a low-pass filter. Such rounding of a waveform becomes inconspicuous when equivalent to or greater than a frequency band of a receiving unit. Specifically, when the frequency band of the receiving unit is denoted by fc, a photoacoustic signal equivalent to a desired signal can be obtained by causing a plurality of light sources to emit beams of light within a time Td satisfying Expression (1) below.

Td≤1/fc (1)

In addition, in order to obtain a more preferable photoacoustic signal, the plurality of light sources may be caused to emit beams of light within a time Td satisfying Expression (2) below.

Td≤1/(2×fc) (2)

In this case, fc is assumed to be a high-frequency side frequency that is lower by 3 dB with respect to sensitivity of a central frequency of the receiving unit.

Furthermore, in order to obtain an even more preferable acoustic signal, Td is favorably set smaller than a period of an A/D conversion rate in order to prevent a low-pass filter from being applied to a digital signal sequence having been subjected to A/D conversion.

Since a photoacoustic signal obtained by emitting beams of light at a time difference equal to or smaller than Td described above is equivalent to a photoacoustic signal obtained by irradiating the object with light of the desired wavelength, in the present embodiment, an operation of simultaneously irradiating beams of light includes a case of irradiating beams of light at a time difference equal to or smaller than Td.

As described above, in the first embodiment, a plurality of wavelengths are selected such that a sum of absorption coefficients of oxyhemoglobin and deoxyhemoglobin at a desired wavelength becomes equal to a sum of absorption coefficients of oxyhemoglobin and deoxyhemoglobin at a wavelength of light to be irradiated.

As a result, a photoacoustic signal equivalent to a photoacoustic signal obtained by irradiating a desired wavelength can be obtained.

Apparatus Configuration

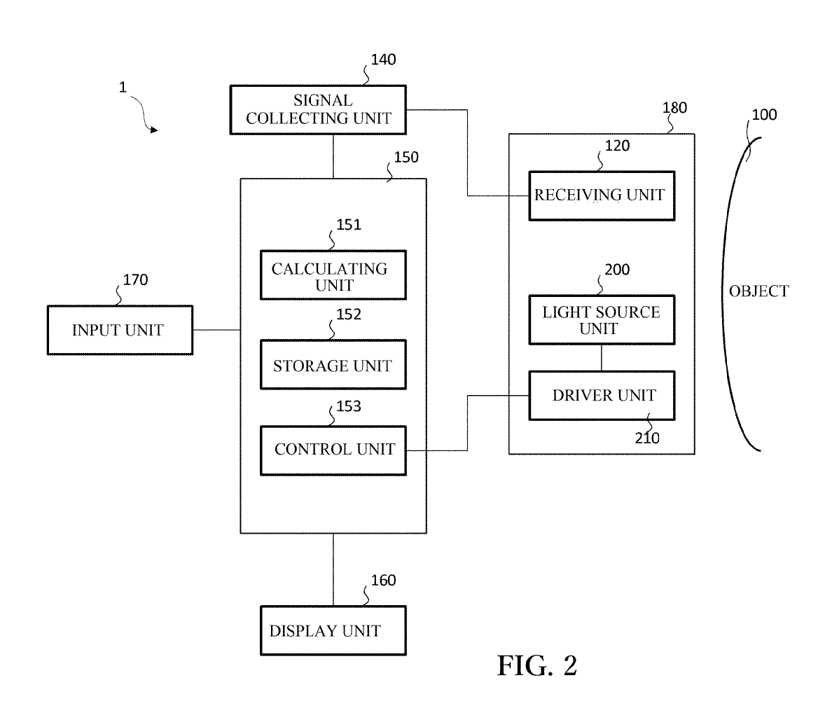

Hereafter, a configuration of a photoacoustic apparatus according to the first embodiment will be described with reference to FIG. 2. The photoacoustic apparatus according to the first embodiment includes a probe 180, a signal collecting unit 140, a computer 150, a display unit 160, and an input unit 170. The probe 180 includes a light source unit 200, a driver unit 210, and a receiving unit 120. The computer 150 includes a calculating unit 151, a storage unit 152, and a control unit 153.

An outline of a measurement method with respect to an object will now be described.

First, the light source unit 200 irradiates an object 100 with pulsed light. In the present embodiment, a plurality of semiconductor light-emitting elements respectively capable of irradiating light of a different wavelength are mounted to the light source unit 200. For example, 16 772-nm laser diodes as light sources corresponding to the wavelength λ1 and 16 945-nm laser diodes as light sources corresponding to the wavelength λ2 are mounted to the probe 180.

Light emission by the plurality of semiconductor light-emitting elements included in the light source unit 200 is controlled by the driver unit 210. As irradiation modes, the driver unit 210 has three modes, namely, a mode in which light sources corresponding to the wavelengths λ1 and λ2 are simultaneously lighted, a mode in which only the light sources corresponding to the wavelength λ1 are simultaneously lighted, and a mode in which only the light sources corresponding to the wavelength λ2 are simultaneously lighted. In the present specification, the modes will be respectively referred to as a mode λ1-λ2, a mode λ1, and a mode λ2.

As described earlier, in the mode λ1-λ2, a photoacoustic signal equivalent to a photoacoustic signal obtained in a case of irradiating light of the wavelength λ0 can be obtained. Therefore, when acquiring structural information of a blood vessel and the like, the mode λ1-λ2 is favorably used.

In addition, when obtaining oxygen saturation that is functional information, a difference between absorption coefficients of oxyhemoglobin and deoxyhemoglobin can be obtained by obtaining photoacoustic signals in the mode λ1-λ2 as well as in at least one of the mode λ1 and the mode λ2. Since methods of acquiring structural information and functional information are well known, a detailed description thereof will be omitted.

When performing a measurement with respect to the object, the semiconductor light-emitting elements included in the light source unit 200 emit beams of pulsed light a plurality of times and irradiate the object 100 with the beams of pulsed light. The receiving unit 120 receives a photoacoustic wave generated from the object 100 in response to each light emission and outputs an analog electrical signal (a photoacoustic signal). The signal collecting unit 140 converts the analog signal output from the receiving unit 120 into a digital signal and outputs the digital signal to the computer 150.

Using the calculating unit 151, the storage unit 152, and the control unit 153, the computer 150 combines digital signals output from the signal collecting unit 140 in response to the respective light emissions and stores the combined digital signals in the storage unit 152 as a combined electrical signal (a photoacoustic signal) derived from photoacoustic waves. It should be noted that the combination is not limited to simple addition and also includes weighted addition, averaging, and moving averaging. Although averaging will be mainly described below as an example, a combination method other than averaging is also applicable. The computer 150 generates photoacoustic data (photoacoustic image data) by performing a reconstruction process and the like on the digital signal stored in the storage unit 152.

The generated photoacoustic image data is displayed by the display unit 160.

Next, details of each component will be described.

### Probe 180

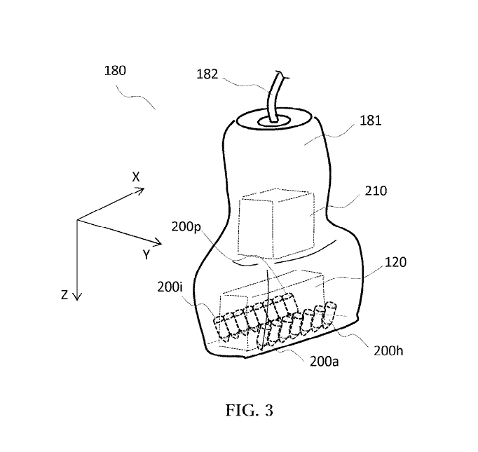

FIG. 3 is a schematic diagram of the probe 180 according to the present embodiment. The probe 180 includes the light source unit 200, the driver unit 210, and a housing 181.

The housing 181 is an enclosure for housing the light source unit 200, the driver unit 210, and the receiving unit 120. By gripping the housing 181, a user can use the probe 180 as a hand-held probe.

As described earlier, the light source unit 200 is realized by mounting a plurality of semiconductor light-emitting elements corresponding to each of a plurality of wavelengths. In the present embodiment, 16 772-nm laser diodes as light sources corresponding to the wavelength λ1 and 16 945-nm laser diodes as light sources corresponding to the wavelength λ2 are mounted to the probe 180. To simplify illustration, FIG. 3 shows an example in which eight each of the laser diodes (200a to 200p) are mounted. Moreover, XYZ axes in the diagram represent coordinate axes when the probe is stationary and are not intended to limit orientations when the probe is in use.

The probe 180 shown in FIG. 3 is connected to the signal collecting unit 140 via a cable 182. The cable 182 includes wiring for supplying power to the light source unit 200, wiring for transmitting a light emission control signal, and wiring for outputting an analog signal output from the receiving unit 120 to the signal collecting unit 140 (all not shown). Alternatively, the cable 182 may be provided with a connector and the probe 180 may be configured so as to be attachable and detachable.

### Light Source Unit 200

The light source unit 200 is means for generating light for irradiating the object 100.

While the light source is desirably a laser light source in order to obtain a large output, a light-emitting diode, a flash lamp, or the like may be used in place of a laser. When using a laser as the light source, various lasers such as a solid-state laser, a gas laser, a dye laser, and a semiconductor laser can be used. Timings, waveforms, intensity, and the like of irradiation are controlled by a light source control unit (not shown). The light source controller may be integrated with the light source.

In addition, when acquiring concentration of a substance such as oxygen saturation, a light source capable of outputting a plurality of wavelengths is favorably used. Furthermore, when mounting the light source unit 200 inside the probe, a semiconductor light-emitting element such as a semiconductor laser or a light-emitting diode is favorably used as shown in FIG. 3.

In order to effectively generate a photoacoustic wave, light must be irradiated in a sufficiently short period of time in accordance with thermal characteristics of the object. When the object is a living organism, a preferable pulse width of the pulse light generated by the light source is around 10 nanoseconds to 1 microsecond. In addition, desirably, a wavelength of the pulse light is a wavelength which enables light to propagate to the inside of the object. Specifically, when the object is a living organism, pulse light of a wavelength of 400 nm or more and 1600 nm or less is desirably used. It is needless to say that the wavelength may be determined in accordance with light absorption characteristics of a light absorber to be imaged.

When imaging a blood vessel at high resolution, a wavelength (400 nm or more and 800 nm or less) which is well absorbed by the blood vessel may be used. In addition, when imaging a deep part of a living organism, light of a wavelength (700 nm or more and 1100 nm or less) which is only weakly absorbed by background tissue (water, fat, and the like) of the living organism may be used.

In the present embodiment, a wavelength of 975 nm which enables structural information of blood vessels to be acquired and which reaches deep parts of an object is selected as the desired wavelength. It is needless to say that other wavelengths can be selected as the desired wavelength.

In the present embodiment, as semiconductor light-emitting elements, 16 each of laser diodes with the wavelength λ1 and laser diodes with the wavelength λ2 are arranged.

In FIG. 3, for the sake of simplicity, laser diodes with the respective wavelengths are mounted in groups of eight (200a to 200p). The laser diodes with the wavelength λ1 (200a to 200h) and the laser diodes with the wavelength λ2 (200i to 200p) oppose each other across the receiving unit 120.

Each laser diode is mounted oriented in a maximum sensitivity direction of the receiving unit 120 so that a distribution of amounts of irradiated light becomes approximately the same in an image region in the mode λ1-λ2. Specifically, as shown in FIG. 3, the laser diodes with the wavelength λ1 (200a to 200h) and the laser diodes with the wavelength λ2 (200i to 200p) are mounted to be angled toward the receiving unit 120.

Moreover, the arrangement of the laser diodes with the wavelength λ1 and the laser diodes with the wavelength λ2 is not limited to this arrangement. In addition, the numbers of the laser diodes with the wavelength λ1 and the laser diodes with the wavelength λ2 are not limited to the exemplified numbers. For example, the laser diodes with the wavelength λ1 and the laser diodes with the wavelength λ2 may be alternately arranged as long as the arrangement enables light of the wavelength λ1 and light of the wavelength λ2 to form approximately equal light amount distributions on the object 100.

In addition, while light sources are arranged so as to oppose each other across the receiving unit 120 in FIG. 3, an arrangement in which the light sources are gathered on one side may be adopted instead. Furthermore, a larger number of semiconductor lasers may be used.

In addition, while a mounting mode in which discrete components are arranged is demonstrated in the example shown in FIG. 3, a plurality of dies cut from a semiconductor wafer may be bonded to a metallic base. Even in this case, the laser diodes with the respective wavelengths may be arranged so that a light amount distribution due to the light of the wavelength λ1 and a light amount distribution due to the light of the wavelength λ2 become approximately the same.

Furthermore, types of semiconductor light-emitting elements are not limited to laser diodes. For example, light-emitting diodes may be used instead. The type and the number of semiconductor light-emitting elements can be determined based on a necessary light amount.

Receiving Unit 120

The receiving unit 120 is a unit constituted by: a transducer (an acoustic wave detecting element) which receives a photoacoustic wave generated due to pulsed light and which outputs an electrical signal; and a supporter which supports the transducer.

A transducer can be used which uses, for example, piezoelectric materials, a capacitive transducer (CMUT), and a Fabry-Perot interferometer as members constituting the transducer. In addition, examples of piezoelectric materials include a piezoelectric ceramic material such as lead zirconate titanate (PZT) and a polymer piezoelectric film material such as polyvinylidene fluoride (PVDF).

An electrical signal obtained by a transducer is a time-resolved signal. In other words, an amplitude of an obtained electrical signal represents a value based on sound pressure (for example, a value proportional to sound pressure) received by the transducer at each time point.

Moreover, as the transducer, a transducer capable of detecting a frequency component (typically, 100 KHz to 10 MHz) constituting a photoacoustic wave is favorably used. In addition, a plurality of transducers may be arranged side by side on the supporter to form a flat surface or a curved surface which is referred to as a 1D array, a 1.5D array, a 1.75D array, or a 2D array.

Furthermore, the receiving unit 120 may include an amplifier for amplifying a time-sequential analog signal output from the transducer. In addition, the receiving unit 120 may include an A/D converter for converting a time-sequential analog signal output from the transducer into a time-sequential digital signal. In other words, the receiving unit 120 may double as the signal collecting unit 140.

Moreover, while a hand-held probe is exemplified in the present embodiment, in order to improve image accuracy, a transducer which surrounds the object 100 from an entire circumference thereof is favorably used so as to enable acoustic waves to be detected from various angles. In addition, when the object 100 is too large to be surrounded from an entire circumference thereof, transducers may be arranged on a hemispherical supporter. When the probe is provided with a receiving unit shaped in this manner, the probe may be mechanically moved relative to the object 100. A mechanism such as an XY stage can be used to move the probe. Moreover, the arrangement and the number of transducers and the shape of the supporter are not limited to those described above and may be optimized in accordance with the object 100.

A medium (an acoustic matching material) that enables a photoacoustic wave to propagate is favorably arranged in a space between the receiving unit 120 and the object 100. Accordingly, acoustic impedance can be matched at an interface between the object 100 and the transducer. Examples of the acoustic matching material include water, oil, and an ultrasonic gel.

In addition, the photoacoustic apparatus according to the present embodiment may include a holding member which holds the object 100 to stabilize a shape of the object 100. A holding member of which light transmittivity and acoustic wave transmittivity are both high is favorable. For example, polymethylpentene, polyethylene terephthalate, acrylic, and the like can be used.

Moreover, when the apparatus according to the present embodiment has a function of generating an ultrasonic image in addition to a photoacoustic image by transmitting and receiving ultrasonic waves, the transducer may be caused to function as transmitting means for transmitting acoustic waves. A transducer as receiving means and a transducer as transmitting means may be a common component or may be separate components.

Signal Collecting Unit 140

The signal collecting unit 140 includes: an amplifier which amplifies an analog electrical signal output from the receiving unit 120; and an A/D converter which converts an analog signal output from the amplifier into a digital signal. The signal collecting unit 140 may be constituted by a field programmable gate array (FPGA) chip or the like.

Analog signals output by a plurality of transducers arranged in an array in the receiving unit 120 are amplified by a plurality of amplifiers corresponding to the respective transducers and converted into digital signals by a plurality of A/D converters corresponding to the respective transducers. A rate of A/D conversion is favorably set to at least twice a band of an input signal or more. As described earlier, when the frequency component constituting a photoacoustic wave ranges from 100 KHz to 10 MHz, the A/D conversion rate is set to 20 MHz or higher and, desirably, 40 MHz or higher.

The signal collecting unit 140 synchronizes a timing of light irradiation and a timing of a signal collection process with each other using a light emission control signal. Specifically, with a light emission time point as a reference, A/D conversion is started at the A/D conversion rate described above to convert an analog signal into a digital signal. As a result, a digital signal sequence can be acquired for each transducer at an interval of a fraction of the A/D conversion rate (a period of an A/D conversion clock). The signal collecting unit 140 is also referred to as a data acquisition system (DAS).

As described earlier, the signal collecting unit 140 may be arranged inside the housing 181 of the probe 180. By adopting such a configuration, since information between the probe 180 and the computer 150 can be propagated using digital signals, noise immunity is improved. In addition, since the number of wirings can be reduced as compared to a case where analog signals are transmitted, operability of the probe 180 is improved. Furthermore, the averaging (to be described later) can also be performed by the signal collecting unit 140. In this case, the averaging is preferably performed using hardware such as an FPGA.

Computer 150

The computer 150 is calculating means which includes the calculating unit 151, the storage unit 152, and the control unit 153. In addition, the computer 150 is also means for controlling the entire photoacoustic apparatus.

A unit which provides a calculation function as the calculating unit 151 can be constituted by a processor such as a CPU or a graphics processing unit (GPU) or an arithmetic circuit such as a field programmable gate array (FPGA) chip. Such units may be constituted by a single processor or a single arithmetic circuit or may be constituted by a plurality of processors or a plurality of arithmetic circuits.

The computer 150 performs the following processes with respect to each of a plurality of transducers.

With respect to a digital signal output from the signal collecting unit 140 for each emission of pulsed light, the computer 150 adds data at a same time point with the light emission time point as a reference to each digital signal and averages the digital signals. In addition, the computer 150 stores the averaged digital signal in the storage unit 152 as an averaged photoacoustic signal.

Furthermore, the calculating unit 151 performs reconstruction of an image based on the (averaged) photoacoustic signal stored in the storage unit 152, and generates a photoacoustic image (a structural image or a functional image) or executes another calculation process. Moreover, the calculating unit 151 may accept input of various parameters related to sound velocity inside the object, a configuration of a holding unit, and the like from the input unit 170 and use the parameters in calculations.

As a reconstruction algorithm used by the calculating unit 151 when converting a photoacoustic signal into a photoacoustic image (for example, three-dimensional volume data), any method such as a time-domain back-projection method, a Fourier domain back-projection method, and a model-based method (a repeat operation method) can be adopted. Examples of a time-domain back-projection method include universal back-projection (UBP), filtered back-projection (FBP), and phasing addition (delay-and-sum).

When the mode is λ1-λ2, the light source unit 200 causes laser diodes corresponding to both the wavelength λ1 and the wavelength λ2 to simultaneously emit beams of light a plurality of times. In addition, the calculating unit 151 obtains an initial sound pressure distribution by an image reconstruction process from an averaged photoacoustic signal. The initial sound pressure distribution is equivalent to an initial sound pressure distribution obtained by irradiating light of the wavelength λ0.

On the other hand, when the mode is λ1, the light source unit 200 only causes the laser diodes of the wavelength λ1 to emit light a plurality of times. In addition, the calculating unit 151 obtains an initial sound pressure distribution corresponding to light of the wavelength λ1 by an image reconstruction process from an averaged photoacoustic signal.

In a similar manner, when the mode is λ2, the light source unit 200 only causes the laser diodes of the wavelength λ2 to emit light a plurality of times. In addition, the calculating unit 151 obtains an initial sound pressure distribution corresponding to light of the wavelength λ2 by an image reconstruction process from an averaged photoacoustic signal.

Furthermore, by correcting the respective obtained initial sound pressure distributions with a light amount distribution, absorption coefficient distributions corresponding to the wavelength λ0, the wavelength λ1, and the wavelength λ2 can be acquired. In addition, an oxygen saturation distribution can be acquired from each absorption coefficient distribution. Moreover, since all that is needed is that an oxygen saturation distribution be eventually obtained, contents and a sequence of the calculations are not limited to the above.

The storage unit 152 is constituted by a volatile memory such as a random access memory (RAM), a read only memory (ROM), and a non-transitory storage medium such as a magnetic disk and a flash memory. Moreover, a storage medium in which a program is to be stored is a non-transitory storage medium. Alternatively, the storage unit 152 may be constituted by a plurality of storage media.

The storage unit 152 is capable of storing various types of data including averaged photoacoustic signals, photoacoustic image data generated by the calculating unit 151, and reconstructed image data based on the photoacoustic image data.

The control unit 153 is means for controlling operations of each component of the photoacoustic apparatus and is constituted by an arithmetic element such as a CPU.

The control unit 153 stores a plurality of irradiation modes, and sends a light emission control signal which controls light emission by a semiconductor light-emitting element to the driver unit 210 in accordance with a specified irradiation mode. In addition, the semiconductor light-emitting element emits light a plurality of times in the specified mode and irradiates an object. As will be described later, the control unit 153 may also have a function for selecting the irradiation mode to be used when acquiring a reconstructed image either in response to an instruction issued by the user or automatically.

In addition, the control unit 153 controls operations of each component of the photoacoustic apparatus by reading a program code stored in the storage unit 152. Furthermore, the control unit 153 performs, for example, adjustment of images output by the display unit 160.

The computer 150 may be an exclusively-designed work station or a general-purpose PC or work station. The computer 150 may be operated according to instructions of a program stored in the storage unit 152. In addition, each component of the computer 150 may be constituted by a different piece of hardware. Alternatively, at least a part of the components of the computer 150 may be constituted by a single piece of hardware.

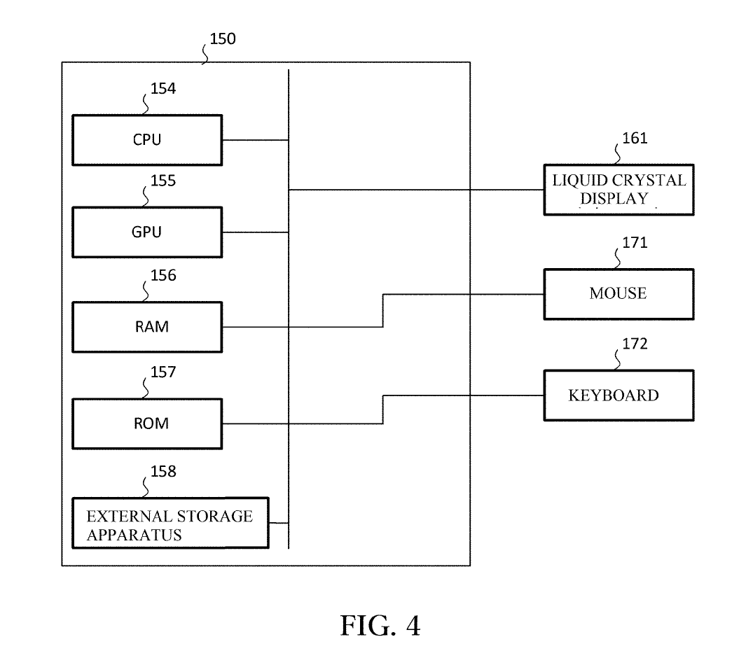

FIG. 4 shows a specific configuration example of the computer 150 according to the present embodiment. The computer 150 according to the present embodiment is configured so as to include a CPU 154, a GPU 155, a RAM 156, a ROM 157, and an external storage apparatus 158. In addition, a liquid crystal display 161 as the display unit 160, and a mouse 171 and a keyboard 172 as the input unit 170 are connected to the computer 150.

The computer 150 and the receiving unit 120 may be configured so as to be housed in a common housing. Alternatively, a part of signal processing may be performed by a computer housed in a housing and remaining signal processing may be performed by a computer provided outside of the housing. In this case, the computers provided inside and outside the housing can be collectively regarded as the computer according to the present embodiment. In other words, hardware constituting the computer may be distributed. Furthermore, as the computer 150, an information processing apparatus provided by a cloud computing service or the like and installed at a remote location may be used.

Moreover, when necessary, the computer 150 may perform image processing or a process of compositing graphic for a GUI with respect to obtained photoacoustic image data.

The user (a physician, a technician, or the like) can carry out a diagnosis by checking the photoacoustic image displayed on the display unit 160. The display image may be stored in a memory inside the computer 150, a data management system connected to the photoacoustic apparatus via a network, or the like based on a storage instruction from the user or the computer 150.

Display Unit 160

The display unit 160 is a display apparatus such as a liquid crystal display and an organic EL. The display unit 160 displays images generated by the computer 150, numerical values at specific positions, and the like. The display unit 160 may also display a GUI for operating images and the apparatus on a screen. In addition, further image processing (adjustment of a brightness value and the like) may be performed on the display unit 160 or the computer 150.

Input Unit 170

The input unit 170 is an operation console constituted by a mouse, a keyboard, and the like which can be operated by the user. Alternatively, the display unit 160 may be constituted by a touch panel, in which case the display unit 160 may be used as the input unit 170. The input unit 170 accepts input of instructions, numerical values, and the like from the user and transmits the input to the computer 150. For example, the user can use the input unit 170 to perform operations such as starting and ending a measurement, specifying an irradiation mode (to be described later), and issuing an instruction to save a created image.

Each component of the photoacoustic apparatus described above may be configured as a separate apparatus or the respective components may be configured as a single integrated apparatus. Alternatively, at least a part of the components of the photoacoustic apparatus may be integrated and the remaining components may be constituted by a separate apparatus.

Object 100

Although the object 100 does not constitute the photoacoustic apparatus according to the present embodiment, a description thereof will be given below. The photoacoustic apparatus according to the present embodiment can be used for the purposes of diagnosing a malignant tumor, a vascular disease, and the like, performing a follow-up observation of chemotherapy, and the like of a human or an animal. Therefore, as the object 100, a diagnostic subject site such as a living organism or, more specifically, breasts, respective internal organs, the vascular network, the head, the neck, the abdominal area, and the extremities including fingers and toes of a human or an animal is assumed. For example, when the measurement subject is a human body, a subject of a light absorber may be oxyhemoglobin, deoxyhemoglobin, a blood vessel containing oxyhemoglobin or deoxyhemoglobin in a large amount, or a new blood vessel formed in a vicinity of a tumor. In addition, the subject of a light absorber may be a plaque on a carotid artery wall or the like. Furthermore, pigments such as methylene blue (MB) and indocyanine green (ICG), gold particulates, or an externally introduced substance which accumulates or which is chemically modified with such pigments or gold particulates may be used as a light absorber. Moreover, a puncture needle or a light absorber added to a puncture needle may be an observation object. The object may be an inanimate matter such as a phantom and a product under test.

Details of Processes

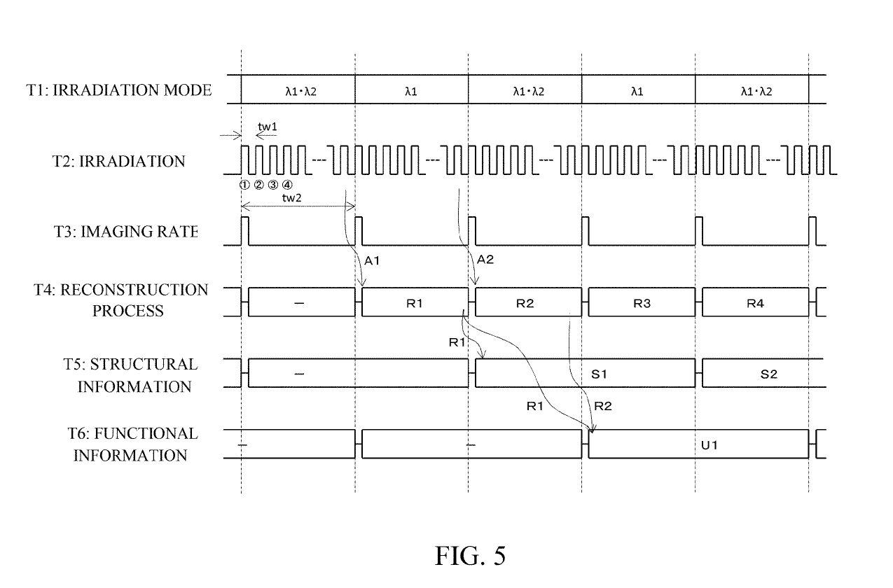

Next, details of processes will be described with reference to FIG. 5 which is a timing chart for explaining operations of the photoacoustic apparatus according to the first embodiment. Note that a horizontal axis in each diagram represents a time axis.

FIG. 5 is a timing chart for explaining, in an easy-to-understand manner, operations according to the first embodiment. In FIG. 5, a horizontal axis represents a time axis. The controls shown are performed by the computer 150 (or an FPGA or dedicated hardware).

In the first embodiment, a photoacoustic signal is acquired using the mode λ1-λ2 and the mode λ1 in combination. Specifically, structural information is acquired based on a photoacoustic signal obtained in the mode λ1-λ2 and, furthermore, functional information is acquired based on both a photoacoustic signal obtained in the mode λ1-λ2 and a photoacoustic signal obtained in the mode λ1.

As shown in T1, the photoacoustic apparatus irradiates pulsed light while periodically switching between irradiation modes of the semiconductor light-emitting elements. The irradiation modes are set so that the mode λ1-λ2 and the mode λ1 are alternately repeated.

Since the light amount of the semiconductor light-emitting elements is small, for the purpose of improving S/N, as shown in T2, the photoacoustic apparatus according to the present embodiment causes the light source unit 200 to repetitively emit light at an irradiation period tw1 and acquires a photoacoustic signal accompanying light emission for each irradiation period tw1. At this point, when the mode is λ1-λ2, the laser diodes of the wavelength λ1 and the wavelength λ2 simultaneously emit beams of light. When the mode is λ1, only the laser diodes of the wavelength λ1 emit light.

Moreover, a length of the irradiation period tw1 may be set in consideration of a maximum permissible exposure (MPE) with respect to skin. For example, when a measurement wavelength is 750 nm, a pulse width of pulsed light is 1 microsecond, and the irradiation period tw1 is 0.1 milliseconds, an MPE value with respect to skin is approximately 14 J/m². On the other hand, when peak power of pulsed light irradiated from a light irradiating unit 113 is 2 kW and an irradiation area from the light irradiating unit 113 is 150 mm², optical energy irradiated to the object 100 is approximately 13.3 J/m². In this case, the optical energy irradiated from the light irradiating unit 113 is equal to or lower than the MPE value.

In this manner, satisfying a condition that the irradiation period tw1 is equal to or longer than 0.1 milliseconds ensures that the optical energy does not exceed the MPE value. As described above, optical energy irradiated to the object can be calculated using a value of the irradiation period tw1, a peak power of pulsed light, and an irradiation area. Moreover, when simultaneously emitting beams of light at two wavelengths, light amounts may be set with sufficient margins.

Let us assume that, during a period in which the mode is λ1-λ2, the laser diodes of the wavelength λ1 and the laser diodes of the wavelength λ2 simultaneously emit beams of light 83 times at the irradiation period tw1.

Subsequently, photoacoustic signals are acquired 83 times at the irradiation period tw1 and averaging is performed, and an averaged photoacoustic signal A1 is acquired for each period (hereinafter, an imaging period) tw2 corresponding to an imaging frame rate.

On the other hand, during a period in which the mode is λ1, the laser diodes of the wavelength λ1 emit light 83 times at the irradiation period tw1. Subsequently, photoacoustic signals are acquired 83 times at the irradiation period tw1 and averaging is performed, and an averaged photoacoustic signal A2 is acquired for each imaging period tw2.

For the averaging, simple averaging, moving averaging, weighted averaging, and the like can be used. When photoacoustic signals are acquired 83 times and the photoacoustic signals are averaged, the imaging period tw2 is 8.3 milliseconds and the imaging frame rate is approximately 120 Hz.

Next, as shown in T4, an image reconstruction process is performed based on the averaged photoacoustic signal A1 to obtain reconstructed image data R1. In addition, an image reconstruction process is performed based on the averaged photoacoustic signal A2 to obtain reconstructed image data R2.

The reconstructed image data is sequentially calculated for each imaging period. In terms of performing a calculation for a reconstruction process, performing a reconstruction process of all regions of an object disadvantageously increases the amount of calculations to be performed. In consideration thereof, for example, in the mode λ1-λ2, only regions approximately uniformly irradiated with both light of the wavelength λ1 and light of the wavelength λ2 may be set as targets of reconstruction. For example, a range having a light amount equal to or more than ½ of a peak light amount may be set as a target of reconstruction.

Subsequently, as shown in T4 to T6, structural information S1 of blood vessels is obtained from the reconstructed image data R1 acquired in the mode λ1-λ2, and oxygen saturation U1 which is functional information is obtained from the reconstructed image data R1 and the reconstructed image data R2 acquired in the mode λ1.

In the present embodiment, structural information can be obtained at a timing of completion of the reconstruction process R1 and functional information can be obtained at a timing of completion of the reconstruction processes R1 and R2. Therefore, by delaying the reconstructed image data of structural information by 8.3 milliseconds, both structural information and functional information can be displayed on the display unit 160 as images with a frame frequency of approximately 60 Hz.

Moreover, when acquiring photoacoustic signals 83 times and averaging the photoacoustic signals, the imaging frame rate becomes approximately 120 Hz which matches a display frame rate of the display unit 160. However, when the numbers of times averaging is performed differs, the imaging frame rate may not match the display frame rate. In this case, a frame rate converter (not shown) may be used to convert the imaging frame rate into the display frame rate.

In addition, although light must be irradiated while switching between a plurality of irradiation modes when acquiring functional information, switching must be completed in a shortest possible time to eliminate the influence of body motion. A long irradiation interval may cause each reconstructed image to deviate and may prevent accurate functional information from being obtained.

As described with reference to FIG. 5, while two irradiation modes λ1-λ2 and λ1 are used in the first embodiment, the types and an order of the irradiation modes are not limited thereto. For example, irradiation may be performed in an order of the mode λ1-λ2, the mode λ1, and the mode λ2. In this case, structural information may be acquired based on a photoacoustic signal obtained by irradiation in the mode λ1-λ2 and functional information may be acquired based on photoacoustic signals obtained by irradiation in the mode λ1 and the mode λ2.

Alternatively, when only structural information is required, only the mode λ1-λ2 may be used.

In addition, while the two wavelengths shown in FIG. 1A are used in the present embodiment, wavelengths to be used are not limited thereto. For example, the wavelengths shown in FIG. 1B may be used or three wavelengths may be used as shown in FIG. 1C.

Furthermore, while an aspect in which the irradiation mode is changed at each arrival of the imaging period tw2 has been demonstrated in the embodiment described above, the irradiation mode may be changed for each irradiation period tw1 or changed when the arrival of the irradiation period tw1 has occurred a prescribed number of times. For example, the irradiation mode may be alternately changed for each irradiation period tw1 and all obtained photoacoustic signals may be averaged. Alternatively, obtained photoacoustic signals for each irradiation mode may be averaged.

As described above, in the first embodiment, by causing beams of light of a plurality of wavelengths that differ from a desired wavelength to be simultaneously irradiated, a photoacoustic signal equivalent to a photoacoustic signal obtained by irradiating light of the desired wavelength can be obtained. As a result, a larger number of options regarding light sources can be provided and a photoacoustic apparatus which prioritizes output and cost can be realized.

### Second Embodiment

In the first embodiment, a signal equivalent to a desired signal is acquired by simultaneously irradiating beams of light of wavelengths that differ from a desired wavelength λ0. In contrast, a second embodiment is an embodiment in which a desired photoacoustic signal is acquired by sequentially irradiating beams of light of wavelengths that differ from a desired wavelength λ0 at different timings and averaging respectively obtained photoacoustic signals.

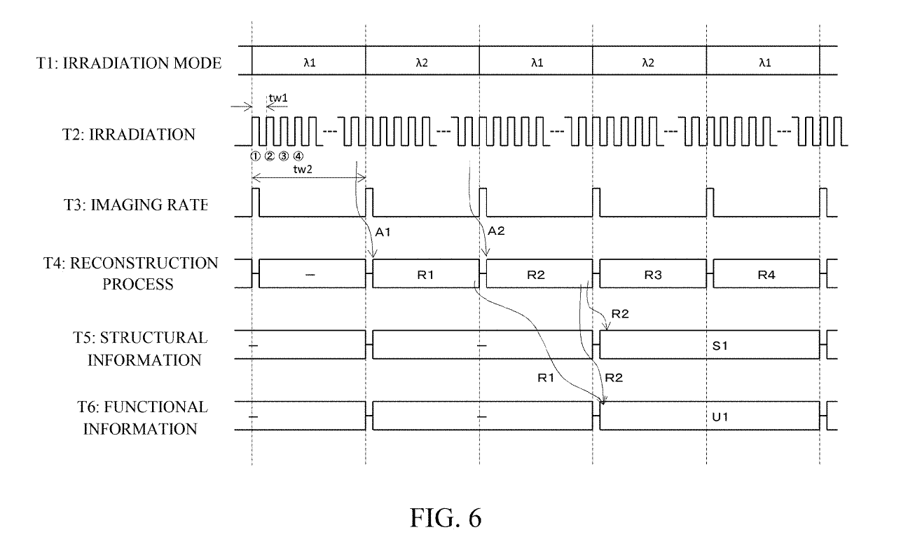

FIG. 6 is a timing chart for explaining, in an easy-to-understand manner, operations by a photoacoustic apparatus according to the second embodiment.

As shown in T1, the photoacoustic apparatus according to the second embodiment irradiates pulsed light while switching between irradiation modes of the semiconductor light-emitting elements. A major difference in the second embodiment from the first embodiment is that irradiation modes differ. In the second embodiment, the mode λ1 and the mode λ2 are set so as to be alternately repeated at intervals of the imaging period tw2.

In the second embodiment, the light source unit 200 is caused to repetitively emit light at the irradiation period tw1 and a photoacoustic signal accompanying light emission is acquired at the irradiation period tw1 in a similar manner to the first embodiment. In doing so, when the mode is λ1, only the laser diodes of the wavelength λ1 emit light. In addition, when the mode is λ2, only the laser diodes of the wavelength λ2 emit light. Moreover, a light amount and a length of the irradiation period tw1 are set within ranges which do not exceed the MPE value with respect to skin in a similar manner to the first embodiment.

In the present example, it is assumed that, during a period in which the mode is λ1, the laser diodes of the wavelength λ1 emit light 83 times at the irradiation period tw1.

Subsequently, photoacoustic signals are acquired 83 times at the irradiation period tw1 and averaging is performed, and an averaged photoacoustic signal A1 is acquired for each imaging period tw2.

In a similar manner, during a period in which the mode is λ2, the laser diodes of the wavelength λ2 emit light 83 times at the irradiation period tw1. Furthermore, photoacoustic signals are acquired 83 times at the irradiation period tw1.

Subsequently, the photoacoustic signals acquired 83 times during the period of the mode λ1 and the photoacoustic signals acquired 83 times during the period of the mode λ2 described above are averaged, and an averaged photoacoustic signal A2 is obtained at a next imaging period.

In this manner, in the second embodiment, by averaging photoacoustic signals obtained by irradiating light of the wavelength λ1 and photoacoustic signals obtained by irradiating light of the wavelength λ2, a signal equivalent to a desired signal can be acquired.

Moreover, in the second embodiment, since two frames worth of time is required to acquire structural information, the imaging frame rate is one-half of 120 Hz (60 Hz).

Next, as shown in T4, an image reconstruction process is performed based on the averaged photoacoustic signal A1 to obtain reconstructed image data R1. In addition, an image reconstruction process is performed based on the averaged photoacoustic signal A2 to obtain reconstructed image data R2.

In the second embodiment, as shown in T4 to T6, structural information S1 of blood vessels is obtained from the reconstructed image data R2 acquired in the mode λ1 and the mode λ2. Furthermore, oxygen saturation U1 which is functional information can be obtained from the reconstructed image data R2 and the reconstructed image data R1 acquired in the mode λ1.

In the second embodiment, both structural information and functional information can be obtained at a period that is twice the imaging period. In other words, both structural information and functional information can be displayed on the display unit 160 as images with a frame frequency of approximately 60 Hz.

As described above, even by averaging photoacoustic signals separately obtained for each wavelength, a photoacoustic signal equivalent to a photoacoustic signal obtained by emitting light of a desired wavelength can be obtained.

In addition, in the second embodiment, photoacoustic signals obtained for each wavelength may be weighted and then combined. By adopting such a configuration, limitations against a plurality of wavelengths can be mitigated.

Moreover, while the two wavelengths shown in FIG. 1A are used in the present embodiment, wavelengths to be used are not limited thereto. For example, the wavelengths shown in FIG. 1B may be used or three wavelengths may be used as shown in FIG. 1C.

Furthermore, while an aspect in which the irradiation mode is changed at each arrival of the imaging period tw2 has been demonstrated in the embodiment described above, the irradiation mode may be changed for each irradiation period tw1 or changed when the arrival of the irradiation period tw1 has occurred a prescribed number of times. For example, the irradiation mode may be alternately changed for each irradiation period tw1 and all obtained photoacoustic signals may be averaged.

In addition, when acquiring functional information, reconstructed image data may be separately acquired using only the mode λ2, and functional information may be calculated from reconstructed image data acquired in the mode λ1 and the reconstructed image data acquired in the mode λ2.

In addition, in a case of averaging photoacoustic signals with each light emission time point as a reference, when photoacoustic waves obtained with light emission time points as a reference are combined in a deviated state, the obtained photoacoustic signal is subjected to a low-pass filter as described in the first embodiment. Therefore, in the second embodiment, desirably, a plurality of light sources are caused to emit light within a time Td satisfying Expression (1) or Expression (2) described earlier and time deviations of photoacoustic waves obtained with light emission time points as a reference are suppressed. Furthermore, a period of the A/D conversion rate is favorably set smaller than Td.

### Third Embodiment

In the first embodiment, a signal equivalent to a desired signal is acquired by simultaneously irradiating beams of light of wavelengths that differ from a desired wavelength λ0. In addition, in the second embodiment, a signal equivalent to a desired signal is acquired by sequentially irradiating beams of light of wavelengths that differ from a desired wavelength λ0.

A third embodiment is an embodiment which shares features of both the first embodiment and the second embodiment.

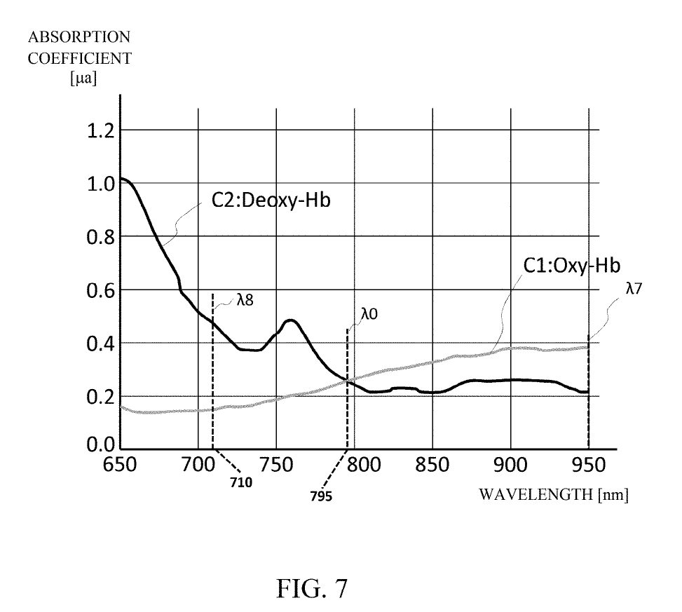

FIG. 7 is a graph showing absorption coefficients of oxyhemoglobin and deoxyhemoglobin for explaining the third embodiment. In FIG. 7, a horizontal axis represents wavelengths of irradiated light and a vertical axis represents absorption coefficients of oxyhemoglobin and deoxyhemoglobin. In FIG. 7, λ0 denoting the desired wavelength is a wavelength of approximately 795 nm at which absorption coefficients of oxyhemoglobin and deoxyhemoglobin are equal to each other.

A wavelength λ7 and a wavelength λ8 represent a plurality of wavelengths which differ from the desired wavelength λ0. In the third embodiment, the following wavelengths are selected as the wavelength λ7 and the wavelength λ8. Specifically, two wavelengths are selected such that a value obtained by adding twice an absorption coefficient at the wavelength λ7 and an absorption coefficient at the wavelength λ8 of oxyhemoglobin is equal to a value obtained by adding twice an absorption coefficient at the wavelength λ7 and an absorption coefficient at the wavelength λ8 of deoxyhemoglobin.

In the present embodiment, the wavelength λ7 is set to 950 nm and the wavelength λ8 is set to 710 nm. Light output of light sources of the two wavelengths is the same. When acquiring a photoacoustic signal using two wavelengths having the relationship described above, an irradiation intensity (or an irradiation time) of light of the wavelength λ7 must be adjusted to twice that of light of the wavelength λ8. By performing irradiation using this method, a photoacoustic signal equivalent to a desired signal can be obtained.

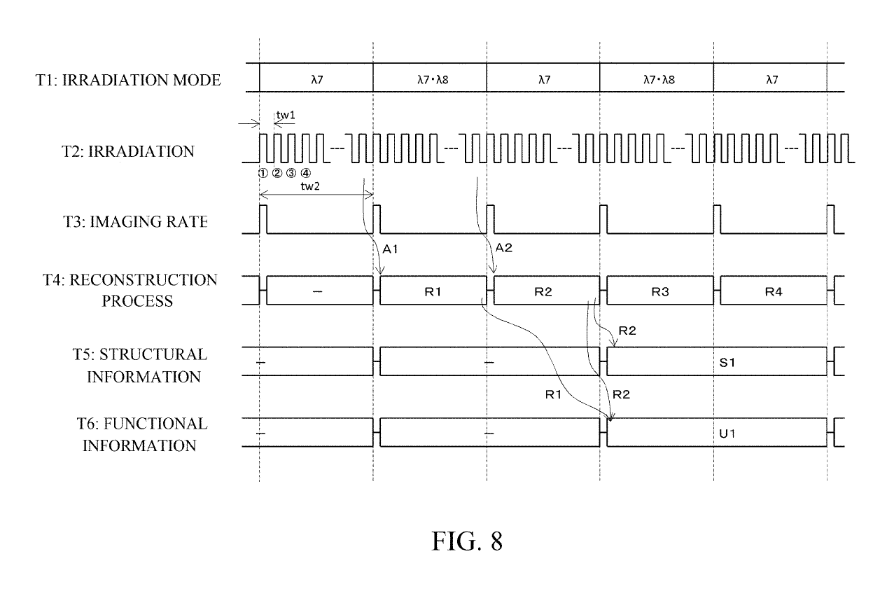

FIG. 8 is a timing chart for explaining, in an easy-to-understand manner, operations by a photoacoustic apparatus according to the third embodiment.

As shown in T1, the photoacoustic apparatus according to the third embodiment irradiates pulsed light while switching between irradiation modes of the semiconductor light-emitting elements. A major difference in the third embodiment from the embodiments described earlier is that irradiation modes differ. In the third embodiment, in a period of a mode λ7, only the laser diodes of the wavelength λ7 are caused to emit light. In addition, in a period of a mode λ7-λ8, beams of light are simultaneously emitted by the laser diodes of the wavelength λ7 and the laser diodes of the wavelength λ8. Furthermore, the period of the mode λ7 and the period of the mode λ7-λ8 are set so as to be alternately repeated at intervals of the imaging period tw2.

In the present example, during a period in which the mode is λ7, the laser diodes of the wavelength λ7 emit light 83 times at the irradiation period tw1 and photoacoustic signals are acquired 83 times at the irradiation period tw1. Subsequently, the photoacoustic signals acquired 83 times during the period of the mode λ7 are averaged, and an averaged photoacoustic signal A1 is obtained at a next imaging period tw2.

On the other hand, during a period in which the mode is λ7-λ8, the laser diodes of the wavelength λ7 and the laser diodes of the wavelength λ8 simultaneously emit beams of light 83 times at the irradiation period tw1 and photoacoustic signals are acquired 83 times at the irradiation period tw1. Subsequently, the photoacoustic signals acquired 83 times during the period of the mode λ7-λ8 and the photoacoustic signals acquired 83 times during the period of the mode λ7 are averaged, and an averaged photoacoustic signal A2 is obtained at a next imaging period tw2. Accordingly, the photoacoustic signal A2 becomes a photoacoustic signal equivalent to a photoacoustic signal obtained by irradiating light of the desired wavelength λ0.

Next, as shown in T4, a process for the image reconstruction described earlier is performed based on the averaged photoacoustic signal A1 to obtain reconstructed image data R1. In addition, a process for the image reconstruction described earlier is performed based on the averaged photoacoustic signal A2 to obtain reconstructed image data R2.

Next, as shown in T4 to T6, structural information S1 of blood vessels is obtained from the reconstructed image data R2. Furthermore, oxygen saturation U1 which is functional information is obtained from the reconstructed image data R1 and the reconstructed image data R2.

In the third embodiment, acquisition periods of the structural information S1 and the functional information U1 are both twice the imaging period. In other words, both structural information and functional information are displayed on the display unit 160 as images with a frame frequency of approximately 60 Hz.

Moreover, while the mode λ7 and the mode λ7-λ8 are repeated in the third embodiment, methods other than the exemplified method may be used as long as the irradiation time of the light of the wavelength λ7 is twice the irradiation time of light of the wavelength λ8. For example, photoacoustic signals obtained by performing irradiation in the mode λ7 twice and irradiation in the mode λ8 once may be averaged. However, as exemplified, by causing a plurality of light sources with different wavelengths to simultaneously emit beams of light, measurement time can be reduced.

### Fourth Embodiment

In the first embodiment, semiconductor light-emitting elements with a plurality of wavelengths that differ from a desired wavelength are caused to simultaneously emit beams of light in order to obtain a photoacoustic signal equivalent to a desired signal. However, in such a case, the following constraints arise with respect to the plurality of selected wavelengths.

For example, in the case of the example shown in FIG. 1A, a value obtained by averaging an absorption coefficient at the wavelength λ1 and an absorption coefficient at the wavelength λ2 of oxyhemoglobin must be equal to a value obtained by averaging an absorption coefficient at the wavelength λ1 and an absorption coefficient at the wavelength λ2 of deoxyhemoglobin.

In addition, in the case of the example shown in FIG. 1C, a value obtained by averaging absorption coefficients at the wavelength λ5, the wavelength λ6, and the wavelength λ7 of oxyhemoglobin must be equal to a value obtained by averaging absorption coefficients at the wavelength λ5, the wavelength λ6, and the wavelength λ7 of deoxyhemoglobin.

In other words, constraints with respect to wavelengths are tight.

A fourth embodiment will now be described in detail using the wavelengths shown in FIG. 7.

In the case of FIG. 7, a value obtained by averaging an absorption coefficient at the wavelength λ7 and an absorption coefficient at the wavelength λ8 of oxyhemoglobin does not equal a value obtained by averaging an absorption coefficient at the wavelength λ7 and an absorption coefficient at the wavelength λ8 of deoxyhemoglobin. In this manner, when using light sources with wavelengths such as those shown in FIG. 7, a photoacoustic signal equivalent to a desired signal cannot be obtained as-is.

In consideration thereof, in the fourth embodiment, the problem described above is solved by changing a light amount of pulsed light for each wavelength.

In the fourth embodiment, the light amount of pulsed light is changed for each wavelength using the three methods described below.

A first method is a method involving changing a light amount of light irradiated from a semiconductor light-emitting element. Specifically, a light amount of light emitted from an element with the wavelength λ1 is set to twice a light amount of light emitted from an element with the wavelength λ8.

By setting light amounts in this manner, the following relationship is satisfied.

Specifically, an average value of an absorption coefficient of oxyhemoglobin x an irradiated light amount at the wavelength λ7 and an absorption coefficient of oxyhemoglobin x an irradiated light amount at the wavelength λ8 equals an average value of an absorption coefficient of deoxyhemoglobin x an irradiated light amount at the wavelength λ7 and an absorption coefficient of deoxyhemoglobin x an irradiated light amount at the wavelength λ8. In other words, a ratio of these values becomes 1:1, which is equal to the ratio of absorption coefficients of oxyhemoglobin and deoxyhemoglobin at the wavelength λ0.

In the first method, a light amount for each wavelength is determined so that a ratio of an average value of absorption coefficients of oxyhemoglobin x irradiated light amounts at a plurality of wavelengths to an average value of absorption coefficients of deoxyhemoglobin x irradiated light amounts at a plurality of wavelengths matches a ratio at the desired wavelength. By adjusting light amounts in this manner, a photoacoustic signal equivalent to a desired signal can be obtained.

A second method is a configuration in which the number of semiconductor light-emitting elements corresponding to each wavelength is changed. Specifically, the number of laser diodes corresponding to the wavelength λ7 is set to twice the number of laser diodes corresponding to the wavelength λ8. Mounting elements as described above enables the light amount of the wavelength λ7 by which the object is irradiated to be readily doubled in a similar manner to the first configuration described above.

Moreover, while an example of changing the number of mounted elements has been described, a similar effect is produced by changing the number of elements which are caused to actually emit light. In the present specification, while simple descriptions of “number of elements” and “number of mounted elements” are used, it is to be understood that these descriptions include the number of light emitting elements which are caused to actually emit light.

A third method is a configuration in which the number of times semiconductor light-emitting elements corresponding to each wavelength is lighted (the number of light emissions performed by semiconductor light-emitting elements corresponding to each wavelength) is changed. Let us now consider a case where the laser diodes of the wavelength λ7 and the laser diodes of the wavelength λ8 are alternately irradiated in a similar manner to the second embodiment. For example, light sources are caused to emit light in the mode λ7 at a first imaging period and a second imaging period, and light sources are caused to emit light in the mode λ8 at a third imaging period. By repeating this process, the light amount of the wavelength λ7 by which the object is irradiated can be doubled. Moreover, while an imaging period (tw2 in the diagram) is used as one unit in the present example, an irradiation period (tw1 in the diagram) may be used as one unit.

As described above, according to the fourth embodiment, constraints with respect to wavelengths can be mitigated by adjusting a light amount of light to be irradiated on the object. In other words, a larger number of options regarding usable wavelengths can be provided.

### Fifth Embodiment

In the fourth embodiment, a light amount of light to be irradiated on an object is controlled for each wavelength. In contrast, the fifth embodiment is a configuration in which a gain with respect to a signal obtained by converting an acoustic wave is changed without changing the light amount of light to be irradiated on an object.

Specifically, a gain with respect to a photoacoustic signal corresponding to the wavelength λ7 is set to twice a gain with respect to a photoacoustic signal corresponding to the wavelength λ8.

Setting gains in this manner enables a similar effect to the fourth embodiment to be produced. Moreover, for example, adjustment of gains may be performed with respect to analog signals using an amplifier or performed with respect to digital signals using a digital multiplier.

According to the fifth embodiment, constraints with respect to wavelengths can be mitigated by adjusting a gain of a signal. In other words, an even larger number of options regarding usable wavelengths can be provided.

### OTHER EMBODIMENTS

It is to be understood that the descriptions of the respective embodiments merely present examples of the present invention and, as such, the present invention can be implemented by appropriately modifying or combining the embodiments without departing from the spirit and the scope of the invention.

For example, the present invention may be implemented as a photoacoustic apparatus which performs at least a part of the processes described above. The present invention may also be implemented as an object information acquiring method which includes at least a part of the processes described above. The processes and units described above may be implemented in any combination thereof insofar as technical contradictions do not arise therefrom.

For example, the plurality of methods exemplified in the fourth and fifth embodiments may be combined with one another. For example, the number of semiconductor light-emitting elements may be changed for each wavelength and, furthermore, a gain with respect to a photoacoustic signal corresponding to each wavelength may be changed.

In addition, while a wavelength at which absorption coefficients of oxyhemoglobin and deoxyhemoglobin are the same has been exemplified as the desired wavelength in the present specification, the desired wavelength may be any wavelength.

Furthermore, a function of transmitting an ultrasonic wave from a transducer and performing a measurement using a reflected wave may be added to the photoacoustic apparatus according to the present embodiment.

In addition, while a photoacoustic apparatus including a hand-held probe has been exemplified in the description of the embodiments, the present invention can also be applied to a photoacoustic apparatus which has a light source such as a solid-state laser and which provides a probe on a stage to perform mechanical scanning. Furthermore, while a photoacoustic apparatus in which a plurality of semiconductor light-emitting elements are mounted inside a hand-held probe has been exemplified in the description of the embodiments, the present invention can also be applied to a photoacoustic apparatus in which a light source such as a solid-state laser is provided outside of a hand-held probe.

In addition, while an example in which a plurality of semiconductor light-emitting elements are caused to emit light a plurality of times and obtained photoacoustic signals are added up has been shown in the description of the embodiments, when a solid-state laser or the like with a large light amount is to be used, the plurality of light emissions and the addition of signals need not necessarily be performed. For example, a configuration may be adopted in which light is emitted once for each imaging period tw2.

In addition, the wavelengths exemplified in the embodiments need not necessarily match the exemplified values.

For example, the wavelength of 772 nm may be around 767 nm to 777 nm and the wavelength of 945 nm may be around 940 nm to 950 nm.

In addition, the wavelength of 783 nm may be around 778 nm to 788 nm and the wavelength of 824 nm may be around 819 nm to 829 nm.

Furthermore, the wavelength of 760 nm may be around 755 nm to 765 nm, the wavelength of 850 nm may be around 845 nm to 855 nm, and the wavelength of 950 nm may be around 945 nm to 955 nm.

Moreover, the effects of the present embodiment can also be produced by using wavelengths included in a range of 755 nm to 950 nm as the plurality of wavelengths that differ from the desired wavelength.

In addition, the wavelength λ0 that is the desired wavelength is a wavelength at which absorption coefficients of oxyhemoglobin and deoxyhemoglobin are equal to each other, and structural information can be obtained in a preferable manner when the wavelength λ0 ranges from 790 nm to 800 nm.

Moreover, in the description of the present embodiment, a light source with a narrow spectrum width has been used as the light source with the plurality of wavelengths that differ from the desired wavelength in order to explain principles in an easy-to-understand manner. However, the light source with the plurality of wavelengths that differ from the desired wavelength which can be used in the present embodiment may be a light source with a wide spectrum width. In this case, the light source with a wide spectrum width may be constituted by a plurality of light sources with a narrow spectrum width, in which case a determination of the number of light emissions and corrections such as addition of signals may be performed based on integral calculations.

The embodiments of the present invention can also be realized by executing the processes described below. Specifically, the present invention can also be realized by supplying a program that realizes one or more functions of the respective embodiments described earlier to a system or an apparatus via a network or various storage media and having one or more processors in a computer in the system or the apparatus read and execute the program. Alternatively, the present invention can also be realized by a circuit (for example, an FPGA or an ASIC) which realizes one or more functions.

According to the present invention, freedom of selection of light sources in a photoacoustic apparatus can be increased.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions. This application claims the benefit of Japanese Patent Application No. 2017-114434, filed on Jun. 9, 2017, which is hereby incorporated by reference herein in its entirety.

What is claimed is:

1. A photoacoustic apparatus, comprising:
a light source configured to simultaneously irradiate an object with lights of a plurality of wavelengths;
acoustic wave detecting unit configured to receive acoustic waves generated from the object being simultaneously irradiated with the lights of the plurality of wavelengths, and convert the acoustic waves into electrical signals; and
signal processing unit configured to acquire characteristics information of the object based on at least the electrical signals.

2. A photoacoustic apparatus, comprising:
a light source configured to respectively irradiate an object with lights of a plurality of wavelengths at different time points;
acoustic wave detecting unit configured to convert a plurality of acoustic waves generated from the object being irradiated with the lights of the plurality of wavelengths, into a plurality of electrical signals; and
signal processing unit configured to acquire characteristics information of the object based on at least a combined electrical signal acquired by combining the plurality of electrical signals.

3. The photoacoustic apparatus according to claim 2, wherein
the combined electrical signal is acquired by performing an averaging process of the plurality of electrical signals.

4. The photoacoustic apparatus according to claim 1, wherein
the object includes a plurality of light absorbers each having a different wavelength dependency of an absorption coefficient, and
when a wavelength at which respective absorption coefficients of the plurality of light absorbers are equal to each other is defined as a reference wavelength, the plurality of wavelengths are different from the reference wavelength.

5. The photoacoustic apparatus according to claim 4, wherein
the object includes a first light absorber and a second light absorber each having a different wavelength dependency of an absorption coefficient, and
the plurality of wavelengths are a combination of wavelengths such that a first ratio being a ratio of a sum of absorption coefficients of the first light absorber to a sum of absorption coefficients of the second light absorber at each of the plurality of wavelengths becomes equal to a second ratio being a ratio of the absorption coefficient of the first light absorber to the absorption coefficient of the second light absorber at the reference wavelength.

6. The photoacoustic apparatus according to claim 4, wherein
the object includes a first light absorber and a second light absorber each having a different wavelength dependency of an absorption coefficient, and
the photoacoustic apparatus further comprises a correcting unit configured to correct a difference between a first ratio being a ratio of a sum of absorption coefficients of the first light absorber to a sum of absorption coefficients of the second light absorber at each of the plurality of wavelengths, and a second ratio being a ratio of the absorption coefficient of the first light absorber to the absorption coefficient of the second light absorber at the reference wavelength.

7. The photoacoustic apparatus according to claim 1, wherein
the plurality of wavelengths are a combination of wavelengths such that a first ratio being a ratio of a sum of absorption coefficients of oxyhemoglobin to a sum of absorption coefficients of deoxyhemoglobin at each of the plurality of wavelengths becomes equal to a second ratio being a ratio of the absorption coefficient of oxyhemoglobin to the absorption coefficient of deoxyhemoglobin at a reference wavelength which differs from any of the plurality of wavelengths.

8. The photoacoustic apparatus according to claim 1, further comprising a correcting unit configured to correct a difference between a first ratio being a ratio of a sum of absorption coefficients of oxyhemoglobin to a sum of absorption coefficients of deoxyhemoglobin at each of the plurality of wavelengths, and a second ratio being a ratio of the absorption coefficient of oxyhemoglobin to the absorption coefficient of deoxyhemoglobin at a reference wavelength which differs from any of the plurality of wavelengths.

9. The photoacoustic apparatus according to claim 6, wherein
the correcting unit is configured to perform the correction by adjusting a sum of irradiation times of each of the lights of the plurality of wavelengths within a prescribed period of time.

10. The photoacoustic apparatus according to claim 6, wherein
the correcting unit is configured to perform the correction by adjusting an irradiated light amount of each of the beams of light of the plurality of wavelengths.

11. The photoacoustic apparatus according to claim 1, wherein
the light source includes a plurality of light emitting elements having been grouped according to wavelength.

12. The photoacoustic apparatus according to claim 10, wherein
the light source includes a plurality of light emitting elements having been grouped according to wavelength, and
the correcting unit is configured to adjust an irradiated light amount with respect to the object by controlling, for each group, light emitting elements to emit light among the plurality of light emitting elements.

13. The photoacoustic apparatus according to claim 10, wherein
the light source includes a plurality of light emitting elements having been grouped according to wavelength, and
the correcting unit is configured to adjust an irradiated light amount with respect to the object by controlling, for each group, the number of times light is emitted by the plurality of light emitting elements.

14. The photoacoustic apparatus according to claim 6, wherein
the correcting unit is configured to perform the correction by adjusting a gain of the electrical signal corresponding to each of the plurality of wavelengths.

15. The photoacoustic apparatus according to claim 4, wherein
the reference wavelength is a wavelength at which absorption coefficients of oxyhemoglobin and deoxyhemoglobin are equal to each other.

16. The photoacoustic apparatus according to claim 4, wherein
the reference wavelength is within a range of 790 nm to 800 nm.

17. The photoacoustic apparatus according to claim 1, wherein
the plurality of wavelengths include:
a first wavelength within a range of 767 nm to 777 nm; and
a second wavelength within a range of 940 nm to 950 nm.

18. The photoacoustic apparatus according to claim 1, wherein
the plurality of wavelengths include:
a first wavelength within a range of 778 nm to 788 nm; and
a second wavelength within a range of 819 nm to 829 nm.

19. The photoacoustic apparatus according to claim 1, wherein
the plurality of wavelengths include:
a first wavelength within a range of 755 nm to 765 nm;
a second wavelength within a range of 845 nm to 855 nm; and
a third wavelength within a range of 945 nm to 955 nm.

20. The photoacoustic apparatus according to claim 1, wherein
the light source is configured to include semiconductor light-emitting elements.
